(19) United States
(12) Patent Application Publication
(10) Pub. No.: US 2026/0053680 A1
(43) Pub. Date: Feb. 26, 2026

(54) ABSORBENT ARTICLE WITH LAMINATE BOND PATTERN

(71) Applicant: The Procter & Gamble Company, Cincinnati, OH (US)

(72) Inventors: Nelson Edward GREENING, II, Cincinnati, OH (US); Tanner Laurie WILLIAMS, Loveland, OH (US); Urmish Popatlal DALAL, Milford, OH (US); Todd Douglas LENSER, Liberty Township, OH (US)

(21) Appl. No.: 19/377,186

(22) Filed: Nov. 3, 2025

Related U.S. Application Data
(63) Continuation of application No. 17/953,364, filed on Sep. 27, 2022.
(60) Provisional application No. 63/250,350, filed on Sep. 30, 2021.

(51) Int. Cl. A61F 13/514; A61F 13/496; A61F 13/513; A61F 13/53

(52) U.S. Cl. CPC A61F 13/51464; A61F 13/496; A61F 2013/51377; A61F 2013/530562

(57) ABSTRACT

An absorbent article including a first waist region, a second waist region, and a crotch region disposed between the first and second waist regions. The absorbent article also includes a chassis having a topsheet, backsheet, and an absorbent core disposed between the topsheet and the backsheet. An elastic laminate may be joined to the chassis in one of the first or second waist regions. The elastic laminate includes an ultrasonically bonded laminate having a first bond pattern and a second bond pattern. The first bond pattern includes a plurality of repeating units and, each repeating unit includes a closed cell unit. The bonds in the first bond pattern include a Bond Separation Distance of about 3.5 mm or less. The first bond pattern is in non-overlapping relationship with the second bond pattern.

### CROSS REFERENCE TO RELATED APPLICATION

This application is a continuation of U.S. patent application Ser. No. 17/953,364, filed on Sep. 27, 2022, which claims the benefit, under 35 U.S.C. § 119 (e), of U.S. Provisional Patent Application No. 63/250,350, filed on Sep. 30, 2021, the entire disclosure each of which are hereby incorporated by reference in their entirety.

### FIELD

The present disclosure relates to absorbent articles having stretch laminates with bond patterns.

### BACKGROUND

Elastomeric laminates are used in various products including absorbent articles (e.g., diapers, incontinence articles, feminine hygiene pads). Such laminates typically include an elastomeric layer that provides extensibility to the laminate and an outer layer that is less stretchable but suitable for providing durability and desirable tactile properties. In this way, the laminate permits a component of an article to closely and comfortably contact the wearer while providing desirable exterior qualities.

Layers of the elastomeric laminate may be combined by various means, including for example thermal bonds in a gathered laminate configuration where corrugations are present in one or more layers. In bonding the layers, manufacturers must balance strength, extensibility, and comfort considerations. These considerations, however, often counteract one another. For example, while more bonds may provide greater lamination strength, it may undermine extensibility. Likewise, more resilient materials may provide greater tear resistance but less softness or breathability.

Therefore, there is a continued need to balance strength, comfort, and extensibility for stretch laminates. There is also a need to provide bond patterns that maximize desired laminate properties while minimizing undesirable traits. Further, there is a need to convey laminate properties through bond patterns.

### SUMMARY

An absorbent article including a first waist region, a second waist region, and a crotch region disposed between the first and second waist regions. The absorbent article includes a chassis having a topsheet, a backsheet, and an absorbent core disposed between the topsheet and the backsheet. An elastic laminate may be joined to the chassis in one of the first or second waist regions. The elastic laminate includes an ultrasonically bonded laminate having a bond pattern including a plurality of repeating units and each repeating unit includes a first closed cell unit. The bonds in the closed cell bond pattern have a Bond Separation Distance of 3.5 mm or less.

An absorbent article including a first waist region, a second waist region, and a crotch region disposed between the first and second waist regions. The absorbent article includes a chassis having a topsheet, backsheet, and an absorbent core disposed between the topsheet and the backsheet. An elastic laminate may be joined to the chassis in one of the first or second waist regions. The elastic laminate includes an ultrasonically bonded laminate having a first coverstock layer, a second coverstock layer, and an elastic film disposed between the first and second coverstock layers. The elastic laminate includes a bond pattern including a plurality of repeating units. Each repeating unit includes a first closed cell unit having a first ratio of a Stretched Enclosed Area to a Relaxed Enclosed Area on the first external surface of at least 1.2.

An absorbent article including a first waist region, a second waist region, and a crotch region disposed between the first and second waist regions. The absorbent article includes a chassis having a topsheet, backsheet, and an absorbent core disposed between the topsheet and the backsheet. An elastic laminate may be joined to the chassis in one of the first or second waist regions. The elastic laminate includes an ultrasonically bonded laminate having a primary substrate and a secondary substrate. The primary substate has a first external surface and an opposing first internal surface. The secondary substrate has a second external surface and an opposing second internal surface. An elastomeric film may be disposed between the primary and secondary substrates. The elastic laminate includes a first bond pattern comprising a Percent Bond Area of at least 3% disposed in a first region. The first bond pattern includes a plurality of closed cell units having a Stretched Enclosed Area to Relaxed Enclosed Area ratio of at least 1.2. The elastic laminate includes second bond pattern disposed in a second region. The first and second regions are non-overlapping, and the second bond pattern is void of closed cell units.

### BRIEF DESCRIPTION OF THE DRAWINGS

The above-mentioned and other features and advantages of the present disclosure, and the manner of attaining them, will become more apparent and the disclosure itself will be better understood by reference to the following description of example forms of the disclosure taken in conjunction with the accompanying drawing.

FIG. 1 is an exploded perspective view of an exemplary laminate according to a nonlimiting embodiment of the present disclosure.

FIG. 2 is a plan view of an exemplary laminate according to a nonlimiting embodiment of the present disclosure.

FIGS. 3A and 3B are schematic representations of exemplary patterns according to nonlimiting embodiments of the present disclosure.

FIGS. 4A and 4B are schematic representations of exemplary patterns according to nonlimiting embodiments of the present disclosure.

FIGS. 5A and 5B are schematic representations of exemplary patterns according to nonlimiting embodiments of the present disclosure.

FIGS. 6A and 6B are schematic representations of exemplary patterns according to nonlimiting embodiments of the present disclosure.

FIG. 7 is a schematic representation of an exemplary pattern according to nonlimiting embodiments of the present disclosure.

FIG. 8 is a schematic representation of an exemplary pattern according to nonlimiting embodiments of the present disclosure.

FIG. 9 is a schematic representation of an exemplary pattern according to nonlimiting embodiments of the present disclosure.

FIG. 10 is a schematic representation of an exemplary pattern according to nonlimiting embodiments of the present disclosure.

FIGS. 11A and 11B are schematic representations of exemplary patterns according to nonlimiting embodiments of the present disclosure.

FIGS. 12A and 12B are schematic representations of exemplary patterns according to nonlimiting embodiments of the present disclosure.

FIG. 13 is a cross-sectional view of a laminate according to a nonlimiting embodiment of the present disclosure.

FIG. 14 is a plan view of the exterior surfaces of a laminate according to a nonlimiting embodiment of the present disclosure.

FIG. 15 is a plan view of an exemplary laminate according to a nonlimiting embodiment of the present disclosure.

FIG. 16 is a plan view of another exemplary laminate according to a nonlimiting embodiment of the present disclosure.

FIG. 17 is schematic plan view of an exemplary absorbent article according to one nonlimiting embodiment of the present disclosure. The absorbent article is shown in a flat, uncontracted state.

FIG. 18A is a schematic perspective view of an exemplary embodiment of an absorbent pant.

FIG. 18B is a schematic plan view of an exemplary embodiment of an absorbent pant precursor structure, prior to joining of the front and rear sections of the belt.

FIG. 19 is a schematic perspective view of a package in accordance with one embodiment of the present disclosure.

FIG. 20 is an illustration of a bond pattern in accordance with one embodiment of the present disclosure.

FIG. 21 is an illustration of a bond pattern in accordance with one embodiment of the present disclosure.

### DETAILED DESCRIPTION

### Definitions

“Absorbent article” refers to devices which absorb and contain body exudates and, more specifically, refers to devices which are placed against or in proximity to the body of the wearer to absorb and contain the various exudates discharged from the body. Exemplary absorbent articles include diapers, training pants, pull-on pant-type diapers (i.e., a diaper having a pre-formed waist opening and leg openings such as illustrated in U.S. Pat. No. 6,120,487), refastenable diapers or pant-type diapers, incontinence briefs and undergarments, diaper holders and liners, feminine hygiene garments such as panty liners, absorbent inserts, and the like.

“Elastic,” “elastomeric,” and “elastically extensible” mean the ability of material, or portion of the material, stretch by at least 30% without rupture or breakage at a given load, and upon release of the load the elastic material or component exhibits at least 70% recovery (i.e., has less than 30% set) in one of the directions as per the Hysteresis Test described herein. Stretch, sometimes referred to as strain, percent strain, engineering strain, draw ratio, or elongation, along with recovery and set may each be determined according to the Hysteresis Test described in more detail below. Materials that are not elastic are referred as inelastic. As used herein, a laminate is elastic if at least 20% of the area of the laminate meets the elastic definition herein. In this situation, the percent of area of the laminate is determined when the laminate is in a fully stretched state.

“Extensible” means the ability to stretch or elongate, without rupture or breakage, by at least 30% as per step 5(a) in the Hysteresis Test herein. As used herein, a laminate is extensible if at least 20% of the area of the laminate meets the extensible definition herein. In this situation, the percent of area of the laminate is determined when the laminate is in a fully stretched state. If a laminate does not meet the definition of elastic above, but does meet the definition of extensible provided in this paragraph, the laminate is an extensible laminate.

“Fully stretched” in reference to a laminate means (1) for corrugated laminates, the laminate is fully stretched when corrugations are substantially flattened by extending the laminate while making sure that the inelastic substrates of the laminate are not plastically deformed, and (2) for laminates without corrugations, the laminate is considered fully stretched without any such extension (i.e., noncorrugated laminates are fully stretched in their relaxed state). “Relaxed” in reference to a laminate means at rest with substantially no external force acting on the laminate, other than gravity.

“Unit” is a smallest building block of a pattern, whose geometric arrangement defines pattern's characteristic imagery and whose repetition in space is necessary to re-construct the pattern. A pattern may be formed from one or more units. A “repeating unit” is a unit that is substantially the same (i.e., slight variation of dimensions, shape, and/or size) or that is the same and is present multiple times within a pattern; said unit may be rotated, mirrored, or otherwise reoriented. The repeating unit is considered substantially the same if its size and/or shape is within 10% of another repeating unit.

“Closed cell unit” means a unit that is identifiable to the human eye with 20/20 vision from 12 inches away as a shape having a perimeter, the perimeter being formed by at least 5 bonds substantially surrounding an area free of permanent bonds that are less than about 3.5 mm Bond Separation Distance per the Bond Measurement Test Method. The perimeter may be formed by discontinuous bonding. For example, discrete bonds that are sufficiently small and/or close together that the viewer sees a shape substantially surrounded by a perimeter. Adjacent bonds along the perimeter of a closed cell unit are no more than about 3.5 mm Bond Separation Distance. Closed cell units may share bond sites with each other to form closed cell.

“Temporary bond” in relation to a laminate means the bond will release upon stretching the laminate by at least 15 mm in the transverse direction from the relaxed state of the laminate. Permanent bonds remain intact upon stretching the laminate by at least 15 mm in the transverse direction from the relaxed state of the laminate.

### Overview

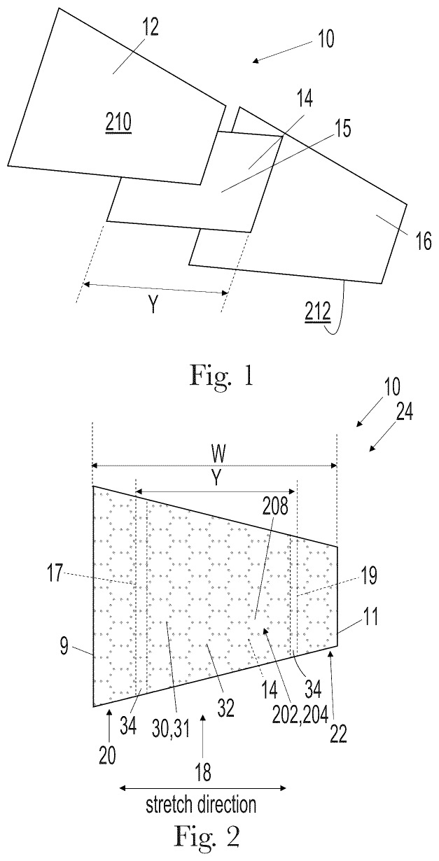

As shown in FIG. 1, a laminate 10 includes a first coverstock layer 12 and an elastomeric layer 14. The laminate may include a second coverstock layer 16, and the elastomeric layer 14 may be sandwiched between the first and second coverstock layers. Coverstock layer materials may be non-elastic. Additional layers may be included (e.g., additional nonwovens, inelastic materials, elastic, or extensible materials, etc.). The laminate may be extensible. In certain embodiments, the laminate is elastomeric. Two or more laminate layers may be joined by a plurality of bonds 30 as shown in FIG. 2. The bonds may be ultrasonic bonds 31, which may join nonwoven layers through the elastomeric layer. An ultrasonically bonded laminate may be formed by any suitable processes, including but not limited to those described in commonly assigned U.S. Pat. Nos. 10,568,775; 10,568,776; and 10,575,993. The bonds may be any suitable shape or size such that two or more laminate layers are joined.

As illustrated in FIG. 2, the laminate includes a first bond pattern 200. The first bond pattern 20 has a first repeating unit 202. The first bond pattern 20 includes closed cell units 204. The closed cell unit of the first bond pattern includes a Stretched Enclosed Area of at least 25 mm² or from about 41 mm² to about 129 mm², according to the Bond Measurement Test Method herein. Additionally, or alternatively, the closed cell unit of the first bond pattern includes a Relaxed Enclosed Area of at least about 19 mm² or from about 19 mm² to about 60 mm² or from about 40 mm² to about 110 mm² or from about 50 mm² to about 120 mm², according to the Extensibility Ratio Test Method herein. Additionally, or alternatively, the closed cell unit of the bond pattern includes a ratio of the Stretched Enclosed Area to the Relaxed Enclosed Area of at least 1.2 or from about 1.2 to about 2 or from about 1.5 to about 2.5. Additionally, or alternatively, the bonds of the first bond pattern may include a Bond Separation Distance of from about 1 mm to about 3.5 mm or from about 1.2 mm to about 3.5 mm or from about 1.5 mm to about 2 mm or less than about 3.5 mm, according to the Bond Measurement Test Method herein. Other patterns may also be present. An ear 130 and/or a waistband 180 of an absorbent article may include a laminate as described herein. These and other features will be described in more detail below.

### Laminate

As noted, the laminate 10 includes one or more coverstock layers 12, 16 and an elastomeric layer 14. Coverstock layer materials may be selected from nonwovens, films and/or any other type of web-based material. In various embodiments, one or more coverstock layers include a nonwoven. Any suitable nonwoven may be used in the laminate 10. Suitable nonwovens may have a basis weight of at least about 8 gsm, or about 30 gsm or less, or about 22 gsm or less, or about 17 gsm or less, or from about 10 gsm to about 22 gsm. Suitable nonwoven include but are not limited to spunbond, spunlaid, meltblown, spunmelt, solvent-spun, electrospun, carded, film fibrillated, melt-film fibrillated, air-laid, dry-laid, wet-laid staple fibers, and other nonwoven web materials formed in part or in whole of polymer fibers, as is known in the art. In nonlimiting examples, a nonwoven includes a meltblown layer. Additionally, or alternatively, a nonwoven may include spunbond layers. In a nonlimiting example, a nonwoven includes two or more spunbond layers. In further nonlimiting examples, one or more nonwovens may include a SMS (spunbond-meltblown-spunbond) configuration. Alternatively, one or more of the nonwovens in the ear of the absorbent article may be void of meltblown layers. While meltblown layers have been found to enhance bonding in ears requiring adhesive (given the meltblown layer's inhibition of the adhesive's diffusion through the porous nonwoven structure), meltblown layers often lack strength. In some embodiments, a nonwoven consists essentially of spunbond layers. In some nonlimiting examples, both the first and the second nonwoven include at least 2 spunbond layers, or 3 or more spunbond layers. The fibers of the nonwoven may be joined by a plurality of thermal point bonds as is known in the art. In non-limiting examples, the nonwoven(s) may include a bond area (with respect to their thermal point bonds) of about 20% or less.

The nonwoven web may be formed predominately of polymeric fibers. In some examples, suitable nonwoven fiber materials may include, but are not limited to polymeric materials such as polyolefins, polyesters, polyamide, nylon, or specifically, polypropylene (PP), polyethylene (PE), poly-lactic acid (PLA), polyethylene terephthalate (PET) and/or blends thereof. In some examples, the fibers may be formed of PP/PE blends such as described in U.S. Pat. No. 5,266,392. Nonwoven fibers may be formed of, or may include as additives or modifiers, components such as aliphatic polyesters, thermoplastic polysaccharides, or other biopolymers. Further useful nonwovens, fiber compositions, formations of fibers and nonwovens and related methods are described in U.S. Pat. Nos. 6,645,569; 6,863,933; and 7,112,621; and in U.S. patent application Ser. Nos. 10/338,603; 10/338,610; and Ser. No. 13/005,237. The individual fibers of a nonwoven layer may be monocomponent or multicomponent (including bicomponent). The multicomponent fibers may be bicomponent, with differing polymeric components in, e.g., a core-and-sheath or side-by-side arrangement. The individual components may include polyolefins such as polypropylene or polyethylene, or their copolymers, or polyesters, thermoplastic polysaccharides, or other biopolymers. Further, the nonwoven may include a blend of different fibers selected, for example from the types of polymeric fibers described above. In some examples, at least a portion of the fibers may exhibit a spiral curl which has a helical shape. According to one example, the fibers may include bicomponent fibers, which are individual fibers each including different materials, usually a first and a second polymeric material. It is believed that the use of side-by-side bi-component fibers is beneficial for imparting a spiral curl to the fibers. Examples of potentially suitable curled or “crimped” bicomponent fibers and nonwovens formed from them are described in U.S. Pat. Nos. 5,382,400; 5,418,045; 5,707,468; 6,454,989; 6,632,386; 5,622,772 and 7,291,239. For purposes herein, use of a nonwoven formed of crimped bicomponent or multicomponent fibers such as, for example, described in the patents and/or patent applications cited immediately above, may be desired as one or both nonwoven layers because they can feel particularly soft to the touch (for wearer comfort on the inside and aesthetically pleasing feel on the outside) and are generally quite pliable. In other nonlimiting examples, a nonwoven may be void of crimped fibers.

Where the laminate 10 includes more than one nonwoven, the nonwovens may have the same basis weight or different basis weights. Likewise, the nonwovens may have the same layer configuration (e.g., SSS) or different layer configurations (e.g., SMS).

The elastomeric layer 14 includes one or more elastomeric materials which provide elasticity to at least a portion of the layer 14. Nonlimiting examples of elastomeric materials include film (e.g., polyurethane films, films derived from rubber and/or other polymeric materials), an elastomeric coating applied to another substrate (e.g., a hot melt elastomer, an elastomeric adhesive, printed elastomer, or elastomer co-extruded to another substrate), elastomeric nonwovens, scrims, and the like. Elastomeric materials can be formed from elastomeric polymers including polymers having styrene derivatives (e.g., styrenic block copolymer materials), polyesters, polyurethanes, polyether amides, polyolefins, combinations thereof or any suitable known elastomers including but not limited to co-extruded VISTAMAXXR. Exemplary elastomers and/or elastomeric materials are disclosed in U.S. Pat. Nos. 8,618,350; 6,410,129; 7,819,853; 8,795,809; 7,806,883; 6,677,258 and U.S. Pat. Pub. No. 2009/0258210. Commercially available elastomeric materials include KRATON (styrenic block copolymer; available from the Kraton Chemical Company, Houston, TX), SEPTON™ (styrenic block copolymer; available from Kuraray America, Inc., New York, NY), VECTOR (styrenic block copolymer; available from TSRC Dexco Chemical Company, Houston, TX), ESTANE® (polyurethane; available from Lubrizol, Inc., Ohio), PEBAX® (polyether block amide; available from Arkema Chemicals, Philadelphia, PA), HYTREL® (polyester; available from DuPont, Wilmington, DE), VISTAMAXX™ (homopolyolefins and random copolymers, and blends of random copolymers, available from EXXON Mobile, Spring, TX), VERSIFY™ (homopolyolefins and random copolymers, and blends of random copolymers, available from Dow Chemical Company, Midland, Michigan), and INFUSE™ (Block copolymer available from Dow Chemical Company).

In nonlimiting examples, the elastomeric layer 14 includes a film 15. The film may be a single layer or multiple layers. The film may be extensible or may be elastic in the lateral direction and/or in the longitudinal direction. The film may be pre-processed, such as by pre-activating, as disclosed, for example, in U.S. Pat. No. 9,533,067. Additionally, or alternatively, the elastomeric layer 14 may be apertured.

The elastomeric layer may be shorter in one or more dimensions of the laminate than the laminate itself. For example, the elastomeric layer may comprise a maximum dimension, Y, in the stretch direction and the laminate may comprise a maximum dimension, W, in the stretch direction. In various embodiments, the stretch direction is the lateral direction. The maximum dimensions are measured when the laminate is in the relaxed state. In nonlimiting examples, Y may be less than W, by at least about 10 mm. In certain embodiments, Y is at least about 20% of, or from about 25% to about 100%, or from about 35% to about 85%, or about 80% or less of W. In various embodiments, the stretch direction is the lateral direction. Additionally, or alternatively, the elastomeric layer may have a dimension that is equal to one or more dimensions of the laminate. For example, the elastomeric layer may comprise substantially the same longitudinal length of the laminate throughout the lateral width of the laminate. In some embodiments, the elastomeric layer may have a basis weight of from about 5 to about 150 gsm, or from about 10 to about 100 gsm, or less than about 150 gsm.

Turning to FIG. 2, the laminate 10 may include a primary region 18, defined by the perimeter of the elastomeric material 14, and one or more inelastic regions 20, 22. The primary region 18 includes an elastic region 32 and one or more unstretched zones 34. In the elastic region, the laminate is elastically extensible. In the unstretched zones, the laminate may not be elastic despite the presence of the elastomeric layer. In some embodiments, the area of the primary region includes at least about 20% of, or from about 30% to about 100%, or about 80% or less of the total area of the laminate. The laminate may include one or more inelastic regions. In certain embodiments, the laminate 10 includes a first inelastic region 20, which extends laterally outward from a first laminate edge 9 of the laminate and is adjacent to the elastic region 32 at a first elastomeric material edge 17. The laminate may include a second inelastic region 22, which may extend laterally inward from a second laminate edge 11 and may be adjacent to the elastic region 18 at a second elastomeric material edge 19. The first and second inelastic regions may be made of the same material(s) or different materials.

In certain embodiments, the laminate 10 includes a gathered laminate 24, wherein one of the layers is strained to a greater degree than a remaining layer during lamination. In this way, the less extensible layer (i.e., the coverstock layer 12, 16) will form gathers when the laminate 24 is in a relaxed state. In some embodiments, at least a portion of the elastomeric layer is strained while the nonwoven(s) are in a relaxed state during lamination. The elastomeric layer may be stretched in one or more directions. Corrugations then form in the nonwoven layer(s) when the subsequently formed laminate 24 is in a relaxed state. When making gathered laminates, the elastomeric layer is stretched in the stretch direction (i.e., the intended direction of stretch in the final product). The stretch direction may be lateral. In nonlimiting examples, the elastomeric layer is stretched in a direction corresponding with the lateral direction of the article. In other words, when the laminate is joined to the chassis subsequent to lamination, the laminate will be oriented such that the laminate is stretchable in the lateral direction of the article (i.e., the laminate is laterally-extensible).

As illustrated in FIG. 2, for example, the laminate layers are joined by one or more bonds 30. Bonds may be any suitable shape and multiple shapes may be utilized within the laminate. In various embodiments, the bonds may be ultrasonic bonds 31. The bonds may be disposed in one or more patterns 200. Each pattern may include one or more closed cell units 204. Repeating closed cell units form one or more repeating units 202. Repeating units are the same or substantially the same closed cell units that repeat in a bond pattern. Thus, a first repeating unit includes first closed cell units having substantially the same shape and a second repeating unit includes second closed cell units having substantially the same shape; however, the first closed cell units and the second closed cell units are different shapes. It is to be appreciated that a closed cell unit of different repeating units may differ in at least one of shape and size. The bond pattern may include closed cell units where each closed cell unit is the same or substantially the same shape and/or size. The bond pattern may include closed cell units where the closed cell units are different, such as different shape and/or size. Certain closed cell units may be the same or substantially the same as certain other closed cell units in the bond pattern and certain other closed cell units may be different. For example, the bond pattern may include closed cell units having only a hexagonal shape, or the bond pattern may include a number of closed cell units having a hexagonal shape and a number of closed cell units having a triangular shape. The laminate may include a first bond pattern 200a, having one or more closed cell units 204 that may repeat. Additionally, or alternatively, the first bond pattern 200 may have a Percent Bond Area of at least about 3%, or from about 3% to about 7%, according to the Bond Measurement Test Method herein. The first bond pattern may at least partially overlap the primary region 18.

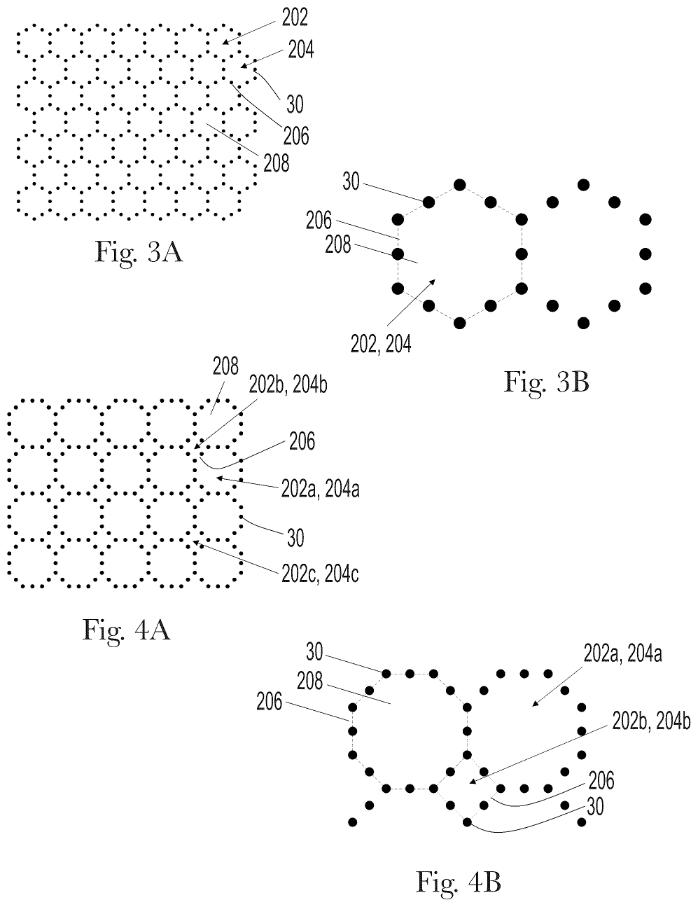

FIGS. 3A-12B provide examples of bond patterns having closed cell units. The closed cell unit includes a perimeter 206 formed by five or more permanent bonds 30. For example, FIGS. 3A and 3B illustrate a closed cell unit 204. FIG. 3A illustrates multiple closed cell units that form a bond pattern, and FIG. 3B illustrates a portion of that bond pattern and the perimeter 206 formed by each of the closed cell units. The perimeter 206 is shown as a dashed-line for illustrative purposes and to better understand the present disclosure; however, the dashed-line forms no part of the bond pattern. The perimeter 206 is formed by connecting adjacent, individual bonds. It is to be appreciated that the perimeter of a first closed cell unit may form part of the perimeter of a second closed cell unit.

As illustrated in FIGS. 4A-6B, for example, a bond pattern may include a first closed cell unit 204a and a second closed cell unit 204b. Similar to the above, the individual, adjacent bonds of the bond pattern may form a perimeter. The first closed cell unit 204a may have a first perimeter and the second closed cell unit 204b may have a second perimeter. The first perimeter may form a part of the second perimeter, or the first perimeter may be separate from the second perimeter. Referring to FIGS. 11A and 11B, for example, a bond pattern may include a first closed cell unit 204a, a second closed cell unit 204b, and a third closed cell unit 204c. Similar to the above, the individual, adjacent bonds of the bond pattern may form one or more perimeters. The first closed cell unit 204a may have a first perimeter, the second closed cell unit 204b may have a second perimeter, and the third closed cell unit 204c may have a third perimeter. Each of the first perimeter, the second perimeter, and the third perimeter may share portions of their perimeters with one another, or each of the first perimeter, the second perimeter, and the third perimeter may be separate. A portion of the perimeter of one closed cell unit 204a may form a portion of the perimeter of another closed cell unit 204b or multiple other closed cell units 204b, 204c.

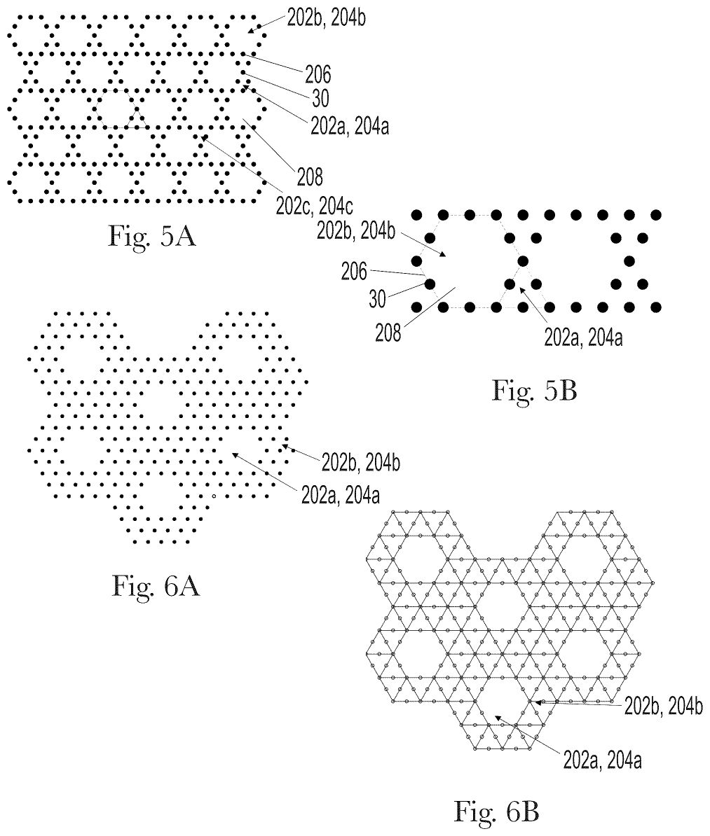

Closed cell units may be any shape such that it is identifiable as a shape having a perimeter that is formed by at least 5 bonds that substantially surround an area. For example, closed cell units may be various shapes including polygons, hearts, circles, ellipses, and combinations thereof. The bond pattern may include closed cell units each having the same shape, such as illustrated in FIG. 3A. The bond pattern may include closed cell units having different shapes, such as illustrated in FIGS. 4A, 5A, 6A, and 11A. Some bond patterns may include more than one closed cell unit 204a, 204b having different shapes. For example, as illustrated in FIGS. 4A and 4B, the bond pattern may include a first closed cell unit 204a and a second closed cell unit 204b. The first closed cell unit 204a may be a hexagon-shaped closed cell unit and the second closed cell unit 204b may be a diamond-shaped closed cell unit. FIGS. 5A and 6A illustrate additional examples of bond patterns having a first closed cell unit and a second closed cell unit having different shapes. As illustrated in FIG. 11A, for example, the bond pattern may include a first closed cell unit 204a, a second closed cell unit 204b, and a third closed cell unit 204c. Each of the first closed cell unit 204a, the second closed cell unit 204b, and the third closed cell unit 204c has a different shape. It is also to be apricated, that various closed cell units may be substantially the same shape but be of different sizes.

One or more portions of the closed cell unit may be formed with a perimeter having individual, discontinuous bond sites, as illustrated, for example, in FIGS. 8-10. The individual bond sites that form the perimeter are not connected. In areas of discontinuous bonding, adjacent bonds along the perimeter of a closed cell unit may have a Bond Separation Distance of about 3.5 mm or less, or about 3 mm or less, or from about 1 mm to about 3.5 mm, according to the Bond Measurement Test Method herein. Further to the above, in areas with discontinuous bonding, the perimeter has at least about 5 bonds, or at least about 6 bonds, or at least about 10 bonds, or from about 5 bonds to about 20 bonds. In nonlimiting examples, bonds may have a Discrete Bond Area of at least about 0.36 mm², or at least about 0.50 mm², or at least about 1.0 mm², or from about 0.36 mm² to about 0.64 mm², or from about 0.38 mm² to about 0.85 mm², or from about 0.40 mm² to about 1.0 mm², or from about 0.45 mm² to about 1.5 mm², or from about 0.5 mm² to about 2 mm², according to the Bond Measurement Test Method herein.

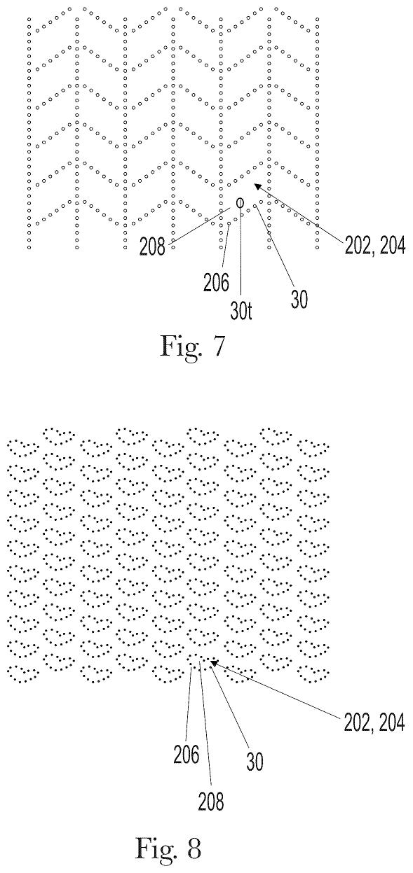

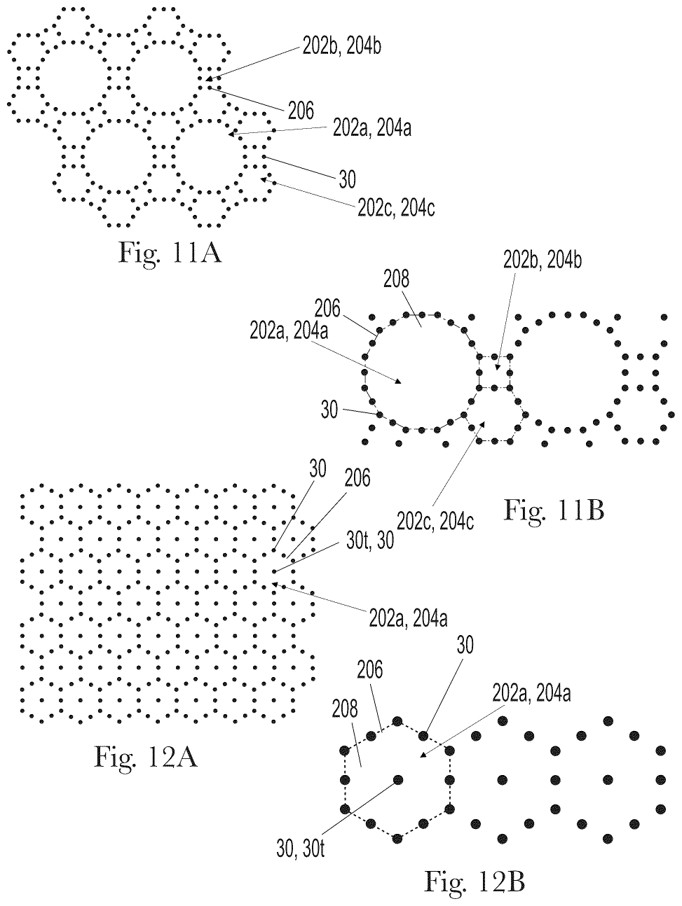

The closed cell unit includes an enclosed portion 208 that is substantially surrounded by the perimeter 206, for example, as illustrated in FIGS. 7 and 8. The closed cell unit may be void of any permanent bonds 30 that are less than 3.5 mm away from every other bond site that forms the closed cell unit. Bonds that are greater than 3.5 mm from the bonds that form the closed cell unit are not part of the closed cell unit. Stated another way, permanent bonds or temporary bonds that are more than 3.5 mm away from the bonds of the closed cell unit form no part of the closed cell unit. For example, as illustrated in FIGS. 12A and 12B, a permanent bond and/or a temporary bond may be placed within the enclosed portion such that the permanent bond and/or temporary bond are more than about 3.5 mm away from the bonds that form the closed cell unit. A temporary bond 30t may be placed within the enclosed portion of the closed cell unit, such as illustrated in FIG. 7. The temporary bond 30t may be at any distance from the permanent bonds that form the closed cell unit. The use of additional bonds within the enclosed portion may allow for the laminate layers to be protected against delamination during processing or handling without undermining the benefits of the closed cell unit. It is to be appreciated that in various embodiments, the enclosed portion of the closed cell unit is void of permanent and temporary bonds.

The closed cell unit may have a Stretched Enclosed Area of at least 35 mm², or at least about 50 mm², or at least about 60 mm², or from about 35 mm² to about 120 mm², or from about 40 mm² to about 90 mm², according to the Bond Measurement Test Method. Where multiple closed cell units are present in a pattern, each closed cell unit may have a different Stretched Enclosed Area. For example, a first closed cell unit 204a has a first enclosed portion and a second closed cell unit has a second enclosed portion, and the first and second enclosed portions may have different Stretched Enclosed Areas. In nonlimiting examples, the second closed cell unit may have a Stretched Enclosed Area that is less than or greater than the Stretched Enclosed Area of the first closed cell unit. It is to be appreciated that each closed cell unit may have substantially the same

### Stretched Enclosed Area.

The closed cell unit may have a Relaxed Enclosed Area of at least 19 mm², or at least about 25 mm², or at least about 40 mm², or from about 19 mm² to about 60 mm², or from about 25 mm² to about 70 mm², or from about 30 mm² to about 100 mm², according to the Extensibility Ratio Test Method. Where multiple closed cell units are present in a pattern, each closed cell unit may have a different Relaxed Enclosed Area. For example, a first closed cell unit 204a has a first enclosed portion and a second closed cell unit 208b has a second enclosed portion, and the first and second enclosed portions may have different Relaxed Enclosed Areas. In nonlimiting examples, the second closed cell unit may have a Relaxed Enclosed Area that is less than or greater than the Relaxed Enclosed Area of the first closed cell unit. It is to be appreciated that each closed cell unit may have substantially the same Relaxed Enclosed Area.

Bond patterns with closed cell units, as discussed herein, allow for relatively larger areas of the nonwoven to gather when the laminate is in a contracted state without the risk of having uncontrolled areas of unbonded regions that could, for example, tear. Further, these bond patterns with closed cell units allow for a more cushion-like feel against the wearer's skin and, likely, would result in less pressure and skin-marking for the wearer. The bond patterns discussed herein also allow for relatively greater breathability by providing more apertures in the film. Further still, the bond pattern provides a visual indication of the amount of stretch as the ear is engaged.

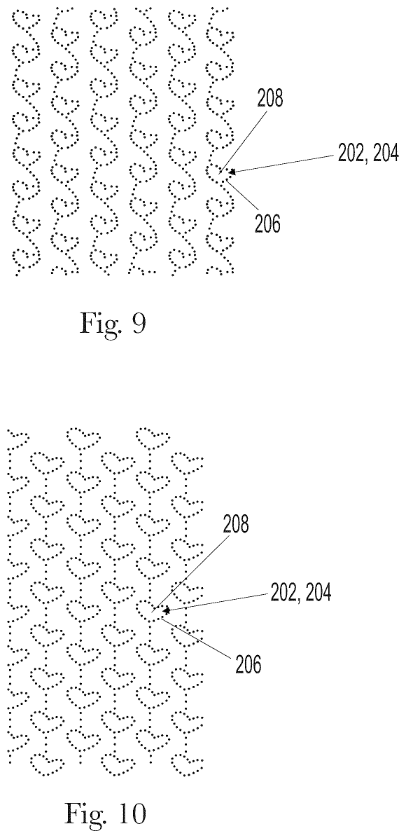

It is also contemplated that the first bond pattern may include closed cell units and areas of the bond pattern that are not enclosed. For example, as illustrated in FIG. 8, the bond pattern may include discrete closed cell units 204. Each of the discrete closed cell units has a perimeter that does not share a perimeter of another closed cell unit. Referring to FIGS. 9 and 10, for example, the bond pattern may include closed cell units and individual bonds disposed in lines. It is also to be appreciated that the bond pattern may include one or more closed cell units that share a perimeter with one another and areas of the bond pattern that are not enclosed.

Additionally, or alternatively, one or more closed cell units may have a Stretched Enclosed Area to Relaxed Enclosed Area ratio of at least about 1.2, or at least about 2, or at least about 2.2, or from about 1.2 to about 2.5. Without being bound by theory, it is believed that units with a Stretched Enclosed Area to Relaxed Enclosed Area ratio within the recited ranges above have a three-dimensional appearance, conveying loft, breathability, and/or cushion to the caregiver and/or wearer while providing enough bonding to ensure continued lamination and integrity. In embodiments where the first bond pattern includes multiple closed cell units, a first closed cell unit may have a first Stretched Enclosed Area to Relaxed Enclosed Area ratio and a second closed cell unit may have a second Stretched Enclosed Area to Relaxed Enclosed Area ratio. The first Stretched Enclosed Area to Relaxed Enclosed Area ratio and the second Stretched Enclosed Area to Relaxed Enclosed Area ratio may be the same. The first Stretched Enclosed Area to Relaxed Enclosed Area ratio and the second Stretched Enclosed Area to Relaxed Enclosed Area ratio may be different. The first Stretched Enclosed Area to Relaxed Enclosed Area ratio may be greater than the second Stretched Enclosed Area to Relaxed Enclosed Area ratio by at least about 5%, or at least about 10%, or at least about 15%, or at least about 20%, or from about 1% to about 50%.

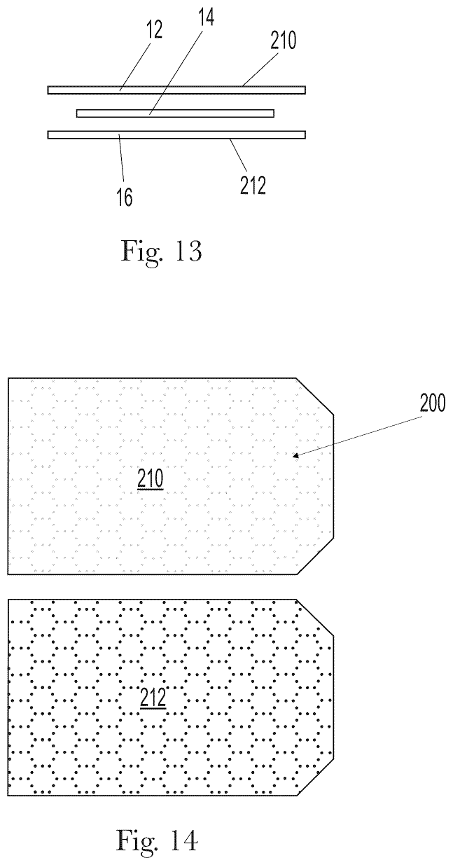

In some embodiments, the laminate may have a first coverstock layer 12 including a first exterior surface 210 and a second coverstock layer 16 including a second exterior surface 212, such as shown in FIG. 13. The first and second coverstock layer may be made from different materials and have different basis weights, layer configurations, and extensibilities. For example, the first coverstock layer includes a nonwoven and the second coverstock layer includes a nonwoven. Alternatively, the first coverstock layer includes a first nonwoven and second coverstock layer includes a second nonwoven, such that the first and second nonwovens differ in at least one of basis weight, layer configuration (e.g., SS, SMS, etc.), and extensibility. In another nonlimiting example, the first and second coverstock layers have the same constituent material but one of the two layers includes a surface treatment or coating causing a change in the material properties such as extensibility, tensile strength, softness, or combinations thereof. In embodiments where the first and second coverstock layer differ, the same bonding pattern 200 may result in different appearance and/or different Stretched Enclosed Area to Relaxed Enclosed Area Ratios on the first and second exterior surfaces 210, 212. As illustrated in FIG. 14, for example, the bonds appear relatively darker and more visually apparent on the second exterior surface 212 than the first exterior surface 210.

The area of the laminate including the first bond pattern may have an Unload Force at 50% of about 0.2 N/in or greater, or about 0.3 N/in or greater, or from about 0.35 to about 1 N/in, reciting for said range every 0.05 N/in increment therein, according to the Hysteresis Test Method herein.

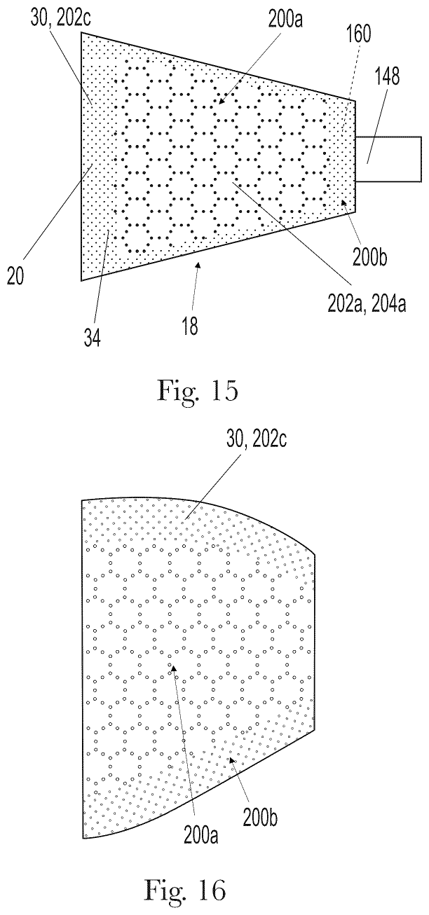

The laminate may include a first bond pattern 200a and a second bond pattern 200b, as illustrated, for example, in FIGS. 15 and 16. The second bond pattern differs from the first bond pattern in at least one of shape of bonds, number of bonds, number of closed cell units (or absence of closed cell units), shape of repeat unit, enclosed area of closed cell units, bond density, bond area, and combinations thereof. The second bond pattern may be positioned outside of the first bond pattern, such that the two patterns may be in non-overlapping relationship. Such as illustrated in FIGS. 15 and 16, the second bond pattern may be disposed along one or more edges of the laminate. In this way, the second bond pattern may at least partially surround or frame at least a portion of the first bond pattern or the entire first bond pattern. The second bond pattern may at least partially overlap an unstretched region 34, an inelastic region 20, a fastening system 148, and/or reinforcement layers 160 (e.g., a folded substrate or additional substrate added to the laminate for enhanced integrity).

The first bond pattern and the second bond pattern may overlap in a transition zone. The overlap of the first bond pattern and the second bond pattern in the transition zone may be less than about 5 mm or less than about 3 mm or less than about 1 mm.

In embodiments with multiple bond patterns, such as a first bond pattern 200a and a second bond pattern 200b, the first bond pattern 200a and the second bond pattern 200b may have different Bond Densities. The first Percent Bond Area may be at least about 3%, or from about 3% to about 7%, according to the Bond Measurement Test Method herein. The second Percent Bond Area may be at least of at least about 4%, or from about 4% to about 10%, according to the Bond Measurement Test Method herein. The Percent Bond Area ratio between patterns may be from about 1.00 to about 3.00, or from about 1.1 to about 2.5 or from about 1.5 to about 2, according to the Bond Dimension Test Method herein. For example, the Percent Bond Area ratio may be about 1.1 or about 1.6.

The second bond pattern 200b may include one or more repeating units 202c. The second bond pattern 200b may be void of closed cell units. Alternatively, the second bond pattern may include one or more closed cell units having any of the features described herein. A closed cell unit in the second bond pattern may include a Stretched Enclosed Area to Relaxed Enclosed Area ratio, less than the Stretched Enclosed Area to Relaxed Enclosed Area ratio of a closed cell unit in the first bond pattern. In nonlimiting examples, the closed cell unit having the largest surface area in the first bond pattern may be larger than the surface area of each of the closed cell units in the second bond pattern. The Stretched Enclosed Area to Relaxed Enclosed Area ratio of the largest first pattern closed cell unit may be at least about 1%, or at least about 5%, or at least about 10%, or at least about 20%, or at least about 50%, or from about 1% to about 50% greater than the Stretched Enclosed Area to Relaxed Enclosed Area ratio of the closed cell unit in the second bond pattern.

In certain embodiments, the laminate may have a Breathability Value of at least about 1 m³/m²/min, or from about 1 m³/m²/min to about 125 m³/m²/min, or from about 2 m³/m²/min to about 50 m³/m²/min, according to the Air Permeability Test Method herein.

Bond pattern of the laminate may coordinate with other portions of the absorbent article. For example, the bond pattern of the laminate may coordinate with a pattern on the chassis, such as the topsheet and/or the backsheet, the ears, the fasteners, and/or the waist feature. The coordinating pattern may be a pattern formed by mechanically changing the structure of the material or adding materials to from a pattern, such as by printing.

### Article Including a Laminate

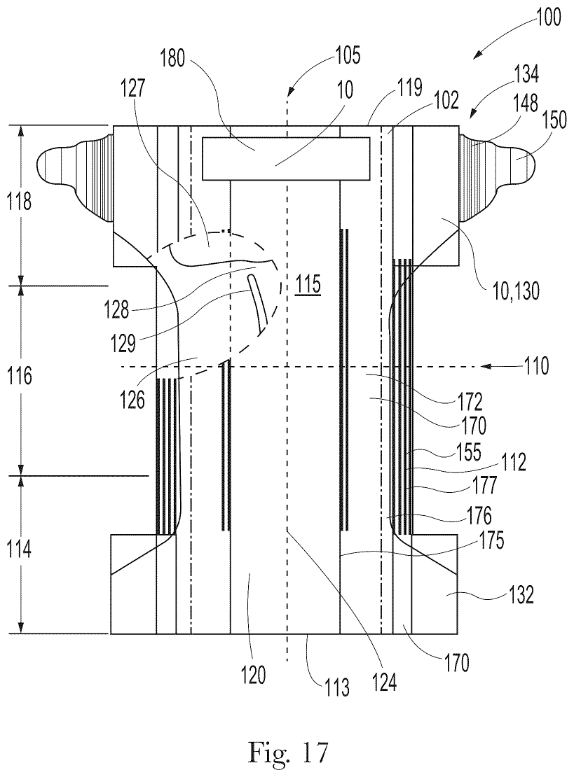

With reference to FIG. 17, a laminate 10 of the present disclosure may be incorporated into an absorbent article 100, such as a disposable absorbent article. The laminate may be attached to one or more layers of the chassis 120 by a chassis attachment bond 102. The chassis attachment bond may include ultrasonic bonds, adhesive bonds, mechanical bonds, or combinations thereof.

FIG. 17 is a plan view of an exemplary, non-limiting embodiment of an absorbent article 100 in a flat, uncontracted state. The body-facing surface 115 of the absorbent article 100 is facing the viewer. The absorbent article 100 includes a longitudinal centerline 105 and a lateral centerline 110.

The absorbent article 100 includes a chassis 120. The absorbent article 100 and chassis 120 are shown to have a first waist region 114, a second waist region 118 opposed to the first waist region 114, and a crotch region 116 located between the first waist region 114 and the second waist region 118. The waist regions 114 and 118 generally include those portions of the absorbent article which, when worn, encircle the waist of the wearer. The waist regions 114 and 118 may include elastic members 155 such that they gather about the waist of the wearer to provide improved fit and containment. The crotch region 116 is the portion of the absorbent article which, when the absorbent article is worn, is generally positioned between the legs of the wearer.

The outer periphery of the chassis 120 is defined by longitudinal edges 112 and waist edges (first waist edge 113 in first waist region 114 and second waist edge 119 in second waist region 118). The chassis 120 may have opposing longitudinal edges 112 that are oriented generally parallel to the longitudinal centerline 105. However, for better fit, longitudinal edges 112 may be curved or angled to produce, for example, an “hourglass” shape article when viewed in a plan view as shown in FIG. 17. The chassis 120 may have opposing lateral edges 113, 119 (i.e., the first waist edge 113 and second waist edge 119) that are oriented generally parallel to the lateral centerline 110.

The chassis 120 may include a liquid permeable topsheet 124, a backsheet 126, and an absorbent core 128 between the topsheet 124 and the backsheet 126. The topsheet 124 may be joined to the core 128 and/or the backsheet 126. The backsheet 126 may be joined to the core 128 and/or the topsheet 124. It should be recognized that other structures, elements, or substrates may be positioned between the core 128 and the topsheet 124 and/or backsheet 126. In some embodiments, an acquisition-distribution system 127 is disposed between the topsheet 124 and the absorbent core 128.

In certain embodiments, the chassis 120 includes the main structure of the absorbent article 100 with other features added to form the composite absorbent article structure. While the topsheet 124, the backsheet 126, and the absorbent core 128 may be assembled in a variety of well-known configurations, absorbent article configurations are described generally in U.S. Pat. Nos. 3,860,003; 5,151,092; 5,221,274; 5,554,145; 5,569,234; 5,580,411; and 6,004,306.

Components of the disposable absorbent article can at least partially include bio-sourced content as described in U.S. Pat. Pub. Nos. 2007/0219521A1, 2011/0139658A1, 2011/0139657A1, 2011/0152812A1, and 2011/0139659A1. These components include, but are not limited to, topsheets, backsheet films, backsheet nonwovens, ears/ear laminates, leg gasketing systems, superabsorbent, acquisition layers, core wrap materials, adhesives, fastener systems, and landing zones. In at least one embodiment, a disposable absorbent article component includes a bio-based content value from about 10% to about 100%, or from about 25% to about 75%, or from about 50% to about 60% using ASTM D6866-10, method B. In order to apply the methodology of ASTM D6866-10 to determine the bio-based content of any component, a representative sample of the component must be obtained for testing. In at least one embodiment, the disposable absorbent article component can be ground into particulates less than about 20 mesh using known grinding methods (e.g., WILEYR mill), and a representative sample of suitable mass taken from the randomly mixed particles.

The laminate 10 of the present disclosure may form or be a portion of one or more components of the article, including but not limited to the ear, waist features, belts, and combinations thereof.

### Topsheet

The topsheet 124 is generally a portion of the absorbent article 100 that may be positioned at least in partial contact or close proximity to a wearer. Suitable topsheets 124 may be manufactured from a wide range of materials, such as porous foams; reticulated foams; apertured plastic films; or woven or nonwoven webs of natural fibers (e.g., wood or cotton fibers), synthetic fibers (e.g., polyester or polypropylene fibers), or a combination of natural and synthetic fibers. The topsheet 124 is generally supple, soft feeling, and non-irritating to a wearer's skin. Generally, at least a portion of the topsheet 124 is liquid pervious, permitting liquid to readily penetrate through the thickness of the topsheet 124. The topsheet 124 may be apertured. The topsheet may be apertured by overbonding a material and then rupturing the overbonds through ring rolling, such as disclosed in U.S. Pat. No. 5,628,097.

Any portion of the topsheet may be coated with a skin care composition, an antibacterial agent, a surfactant, and/or other beneficial agents. The topsheet may be hydrophilic or hydrophobic or may have hydrophilic and/or hydrophobic portions or layers. If the topsheet is hydrophobic, typically apertures will be present so that bodily exudates may pass through the topsheet.

### Absorbent Core

The absorbent core 128 may include a wide variety of liquid-absorbent materials commonly used in disposable diapers and other absorbent articles. Examples of suitable absorbent materials include comminuted wood pulp, which is generally referred to as air felt creped cellulose wadding; melt blown polymers, including co-form; chemically stiffened, modified, or cross-linked cellulosic fibers; tissue, including tissue wraps and tissue laminates; absorbent foams; absorbent sponges; superabsorbent polymers; absorbent gelling materials; or any other known absorbent material or combinations of materials. In one embodiment, at least a portion of the absorbent core is substantially cellulose free and contains less than 10% by weight cellulosic fibers, less than 5% cellulosic fibers, less than 1% cellulosic fibers, no more than an immaterial amount of cellulosic fibers or no cellulosic fibers. It should be understood that an immaterial amount of cellulosic material does not materially affect at least one of the thinness, flexibility, and absorbency of the portion of the absorbent core that is substantially cellulose free. Among other benefits, it is believed that when at least a portion of the absorbent core is substantially cellulose free, this portion of the absorbent core is significantly thinner and more flexible than a similar absorbent core that includes more than 10% by weight of cellulosic fibers. The amount of absorbent material, such as absorbent particulate polymer material present in the absorbent core may vary, but in certain embodiments, is present in the absorbent core in an amount greater than about 80% by weight of the absorbent core, or greater than about 85% by weight of the absorbent core, or greater than about 90% by weight of the absorbent core, or greater than about 95% by weight of the core. In some embodiments, the absorbent core may include one or more channels 129, wherein said channels are substantially free of absorbent particulate polymer material. The channels 129 may extend longitudinally or laterally. The absorbent core may further include two or more channels. The channels may be straight, curvilinear, angled or any workable combination thereof. In nonlimiting examples, two channels are symmetrically disposed about the longitudinal axis.

### Backsheet

The backsheet 126 is generally positioned such that it may be at least a portion of the garment-facing surface of the absorbent article 100. The backsheet 126 may be joined to portions of the topsheet 124, the absorbent core 128, and/or any other layers of the absorbent article by any attachment methods known to those of skill in the art. Backsheet 126 may be designed to prevent the exudates absorbed by and contained within the absorbent article 100 from soiling articles that may contact the absorbent article 100, such as bed sheets and undergarments. In certain embodiments, the backsheet 126 is substantially water-impermeable. The backsheet may, for example, be or include a thin plastic film, such as a thermoplastic film having a thickness of about 0.012 mm to about 0.051 mm. Other suitable backsheet materials may include breathable materials which permit vapors to escape from the absorbent article, while still preventing, or at least inhibiting, bodily exudates from passing through the backsheet.

Backsheet 126 may also consist of more than one layer. The backsheet 126 may include an outer cover and an inner layer. The outer cover material 40 may include a bond pattern, apertures, and/or three-dimensional features. The outer cover material 40 may be a nonwoven material, such as a hydroentangled nonwoven material. The inner layer may be made of a substantially liquid-impermeable film, such as a polymeric film. The outer cover and an inner layer may be joined together by adhesive or any other suitable material or method.

### Ears/Fasteners

The absorbent article 100 may include one or more ears 130, including for example front ears 132 disposed in the first waist region and/or back ears 134 disposed in the second waist region. The ears 130 may be integral with the chassis or discrete elements joined to the chassis 120 at a chassis attachment bond 102, which may join one or more layers of the ear to the chassis. The ears 130 may be extensible or elastic. The ears 130 may be formed from one or more nonwoven webs, woven webs, knitted fabrics, polymeric and elastomeric films, apertured films, sponges, foams, scrims, or combinations and/or laminates of any the foregoing.

In some embodiments, the ear 130 may include elastomers, such that the ear is stretchable. In certain embodiments, the ears 130 may be formed of a stretch laminate such as a nonwoven/elastomeric material laminate or a nonwoven/elastomeric material/nonwoven laminate, which also results in the ear being stretchable. The ear 120 may be extensible in the lateral direction of the article. In some embodiments, the ear is elastic in the lateral direction. In further embodiments, the ear 130 may extend more in the lateral direction than in the longitudinal direction. Alternatively, the ear may extend more in the longitudinal direction than in the lateral direction. In certain nonlimiting examples, the ear may include one or more inelastic regions along with a separate elastic region.

In some embodiments, the ear includes a laminate of one or more nonwovens and one or more elastic materials, such as the laminate 10 having any of the features or laminate layers described herein.

Any suitable nonwoven may be used in an ear 130. Suitable nonwovens may have a basis weight of at least about 8 gsm, or less than about 30 gsm, or about 17 gsm or less, or from about 10 gsm to about 17 gsm. Typically, lower basis weight nonwovens reduce an ear's overall strength. However, ears designed according to the principles herein can obtain high strength despite lower basis weight nonwovens. Where the ear 130 includes more than one nonwoven, the nonwovens may have the same basis weight or different basis weights. Likewise, the nonwovens may have the same layer structure or different layer structures. Further, a nonwoven in the ear may have the same or different features of nonwovens in the backsheet, topsheet, leg gasketing system and/or waist feature.

The ear may be an ultrasonically bonded ear as is disclosed for example in U.S. patent application Ser. No. 15/674,559. The ear may be a gathered laminate 24. Alternatively, the ear may be activated by processes disclosed in U.S. Pat. Pub. No. 2013/0082418, U.S. Pat. Nos. 5,167,897; 5,993,432; 5,156,793; 5,167,897; 7,062,983 and 6,843,134 for example.

The ear may be joined to the chassis at a chassis attachment bond 102. In some nonlimiting examples, the chassis attachment bond is located in an inelastic region of the ear.

The absorbent article 100 may also include a fastening system 148. When fastened, the fastening system 148 interconnects the first waist region 114 and the rear waist region 118 resulting in a waist circumference that may encircle the wearer during wear of the absorbent article 100. The fastening system 148 may include a fastening elements 150 such as tape tabs, hook and loop fastening components, interlocking fasteners such as tabs & slots, buckles, buttons, snaps, and/or hermaphroditic fastening components, although any other known fastening means are generally acceptable. The absorbent article may include a landing zone to which a fastening element can engage and/or a release tape that protects the fastening elements from insult prior to use. Some exemplary surface fastening systems are disclosed in U.S. Pat. Nos. 3,848,594; 4,662,875; 4,846,815; 4,894,060; 4,946,527; 5,151,092; and 5,221,274. An exemplary interlocking fastening system is disclosed in U.S. Pat. No. 6,432,098. In some embodiments, the fastening system 148 and/or the element 150 is foldable.

The fastening system 148 may be joined to any suitable portion of the article 100 by any suitable means. The fastening system may be joined to the ear between layers.

### Leg Gasketing System

The absorbent article 100 may include a leg gasketing system 170 attached to the chassis 120, which may include one or more cuffs. The leg gasketing system may include a pair of barrier leg cuffs 172. Each barrier leg cuff may be formed by a piece of material which is bonded to the absorbent article so it may extend upwards from a wearer-facing surface of the absorbent article and provide improved containment of fluids and other body exudates approximately at the junction of the torso and legs of the wearer. The barrier leg cuffs are delimited by a proximal edge joined directly or indirectly to the topsheet 124 and/or the backsheet 126 and a free terminal edge 175, which is intended to contact and form a seal with the wearer's skin. In some embodiments, the free terminal edge 175 includes a folded edge. The barrier leg cuffs 172 extend at least partially between the front waist edge 113 and the rear waist edge 119 of the absorbent article on opposite sides of the longitudinal centerline 105 and are at least present in the crotch region. The barrier leg cuffs may be joined at the proximal edge with the chassis of the article by a bond which may be made by gluing, fusion bonding, or a combination of other suitable bonding processes.

The barrier leg cuffs may be integral with the topsheet 124 or the backsheet 126 or may be a separate material joined to the article's chassis. Each barrier leg cuff 172 may include one, two or more elastic elements 155 close to the free terminal edge 175 to provide a better seal.

In addition to the barrier leg cuffs 172, the article may include gasketing cuffs 176, which are joined to the chassis of the absorbent article, in particular to the topsheet 124 and/or the backsheet 126 and are placed externally relative to the barrier leg cuffs 172. The gasketing cuffs 176 may provide a better seal around the thighs of the wearer. A gasketing cuff may include a proximal edge and a free terminal edge 177. The free terminal edge 177 may include a folded edge. Each gasketing cuff may include one or more elastic elements 155 in the chassis of the absorbent article between the topsheet 124 and backsheet 126 in the area of the leg openings. All, or a portion of, the barrier leg cuffs and/or gasketing cuffs may be treated with a lotion or another skin care composition.

In further embodiments, the leg gasketing system includes barrier leg cuffs that are integral with gasketing cuffs. Suitable leg gasketing systems which may be part of the absorbent article are disclosed in U.S. Pat. App. No. 62/134,622, 14/077,708; U.S. Pat. Nos. 8,939,957; 3,860,003; 7,435,243; 8,062,279.

### Elastic Waist Feature

The absorbent article 100 may include at least one elastic waist feature 180 that helps to provide improved fit and containment, as shown in FIG. 17. The elastic waist feature 180 is generally intended to expand and contract to dynamically fit the wearer's waist. Elasticized waist features include waistbands, waist cuffs having pockets formed from a portion of the waist feature 180 that is unattached from the chassis 120, and waist panels designed to fit securely about the abdomen of the wearer. Nonlimiting examples of elasticized waist features are disclosed in U.S. patent application Ser. Nos. 13/490,543; 14/533,472; and 62/134,622. Waist features 180 may be joined to the chassis 120 in the first waist region 114 and/or in the second waist region 118. The waist feature can be used in conjunction with the ear 130 to provide desirable stretch and flexibility for proper fit of the article on the wearer. The waist feature may include a laminate 10 having any of the features described herein. The waist feature may be extensible or elastic in the lateral and/or longitudinal directions. In some embodiment, the waist feature 180 includes a belt 220. The waist feature may be attached to the chassis at a waist feature bond 182.

### Adult or Baby Pant Absorbent Articles

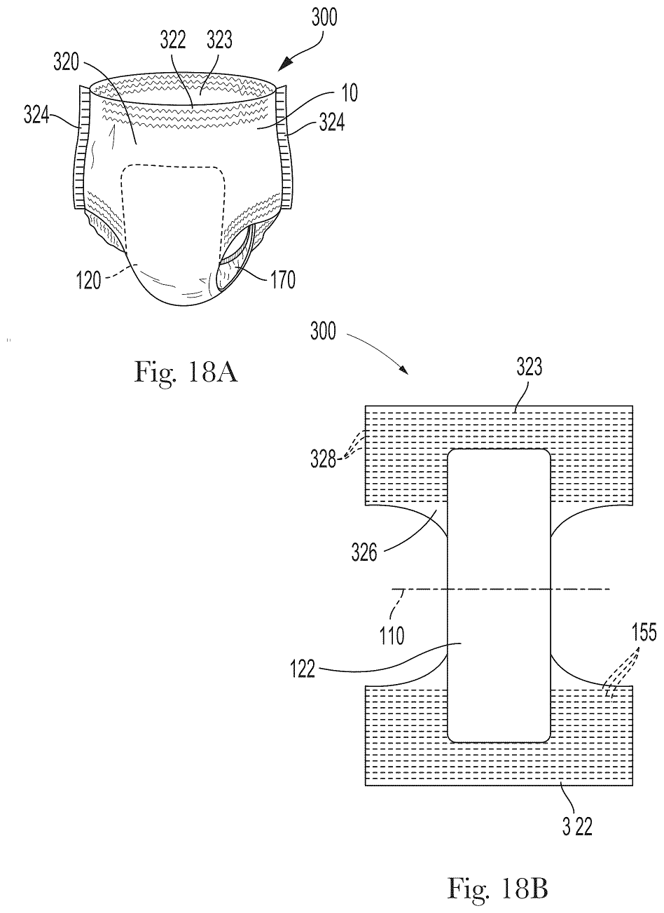

In some embodiments, the article 100 may include an absorbent pant 300 as shown in FIGS. 18A and 18B. The absorbent pant may include a chassis 120, a belt 320 to be positioned about the wearer's waist, and optionally a leg gasketing system 170. FIG. 18B depicts an exemplary precursor structure of the pant in FIG. 18A, in an open configuration laid out flat and stretched out laterally against elastic-induced contraction. In the final assembly of the pant, the front belt portion 322 is joined to rear belt portion 323 at seams 324, which may be permanent or refastenable. To form the pant 300, the precursor structure may be folded at or about lateral centerline 110 with the topsheet 124 facing inward, and the longitudinal edges of the front 322 and rear 323 belt portions may be joined at seams 324, forming a pant structure having leg openings, front waist edge and rear waist edge. In this way, the pant 300 may include a pre-formed, continuous waist opening and pre-formed, continuous leg openings for the wearer at the time of donning the pant 300.

The front and rear belt portions 322, 323 may be the outermost structures forming the front and rear regions of a pant 300. The pant may include an outer wrap 326 wrapping the entirety of the front, crotch, and rear regions, and forming an outermost pant-shaped structure. In some embodiments, the outer cover of the backsheet forms the outer wrap. An outer wrap 326 may be formed of one or more sections of nonwoven web and may be cut to a profile providing suitably tailored leg opening edge profiles as desired.

A belt 320 may include the laminate 10 of the present disclosure, having any of the afore-described features including one or more nonwoven layers and one or more elastomeric layers. The laminate layers may be joined by ultrasonic bonding.

According to some nonlimiting examples, the nonwoven used for a belt portion may include a material that provides good recovery when external pressure is applied and removed.

Elastomeric layers of waist features, such as belt portions, may include one or more elastic members 155. The elastic members 155 may be elastomeric fibers, such as LYCRAR fibers available from INVISTA of Wichita, KS, in various decitex levels. The elastic members 155 may also include any heat shrinkable elastic material as is well known in the art. Other suitable elastics can be made with various other materials including but not limited to: rubbers, styrene ethylbutylene styrene, styrene ethylene propylene styrene, styrene ethylene propylene styrene, styrene butadiene styrene, styrene isoprene styrene, polyolefin elastomers, elastomeric polyurethanes, and other elastomeric materials known in the art, and combinations thereof. In some nonlimiting examples, the elastic members may be extruded strand elastics with any number of strands (or filaments). In some embodiments, the elastic members can have a decitex ranging from 50 to 2000, or any integer value for any decitex value in this range. However, the skilled person may select the appropriate decitex based on the desired contraction and other principles discussed herein. In further embodiments, the elastic members may be in a form of film. Examples of films have been described in prior patent applications (see, for example, U.S. Pat. App. Pub. No. 2010/0040826). The film may be created with a variety of resins combined in at least one of several sublayers, the latter providing different benefits to the film.

In addition, elastic members 155 may take a multitude of configurations. For example, the width may be varied; a single strand or several parallel or non-parallel strands of elastic material may be used; or a variety of shapes may be used including rectilinear and curvilinear; or a variety of cross sectional shapes can be used (circular, rectangular, square, etc.).

Layers of a waist feature (e.g., belt portion) and/or chassis 120 may be joined together about elastic strands 155 by adhesive deposited between the layers, by thermal bonds, by compression bonds, or by a combination thereof. In other examples, the one or more elastic members may be strips or a section of film formed of elastomeric material. Where the elastic member is elongate, it may be desirable that the longer dimension be laterally oriented, or even substantially aligned with the lateral direction, as strands 155 are depicted in FIG. 18B for example.

A belt portion or other form of waist feature may include at least 3 waist elastic members, at least 5 elastic members, at least 10 waist elastic members, or at least 15 waist elastic members, or from about 2 to about 35 waist elastic members, or from about 5 to about 25 waist elastic members.

In one embodiment, adjacent elastic members 155 are spaced a longitudinal distance of at least 3.5 mm apart from one edge of the member to the other edge of the member, optionally at least 4 mm apart; optionally at least 4.5 mm apart; optionally at least 5 mm apart; optionally at least 5.5 mm apart; optionally at least 6 mm apart; optionally at least 6.5 mm apart; optionally at least 7 mm apart; optionally at least 7.5 mm apart; optionally at least 8 mm apart; optionally at least 8.5 mm apart; optionally at least 9 mm apart; optionally at least 9.5 mm apart; optionally at least 10 mm apart; optionally at least 10.5 mm apart; optionally at least 11 mm apart; optionally at least 11.5 mm apart; optionally at least 12 mm apart. The spacing between elastic members may be the same or different across the longitudinal length of the waist feature. For example, the spacing between adjacent elastic members could uniformly be 7 mm or there could be variable spacing (i.e., two adjacent elastic members are separated by 3 mm, another two are separated by 6.5 mm, etc.).

During manufacture of the waist feature, the elastic members 155 may be pre-strained by a desired amount as they are being incorporated into the waist feature. Upon subsequent relaxation of the waist feature, the elastic members will contract laterally toward their unstrained lengths. This may cause layers of the waist feature to gather and form ruffles or rugosities having ridges and valleys generally transverse to the lengths of the elastic members 155 and extending in the z-direction.

In further embodiments, to adhere the components of the waist feature laminate, the elastic members may be individually coated with adhesive (“strand coated”) prior to incorporation into the waist laminate. Various coating methods and techniques, including strand coating methods and techniques, are shown for example in U.S. Pat. Nos. 5,340,648; 5,501,756; 5,507,909; 6,077,375; 6,200,635; 6,235,137; 6,361,634; 6,561,430; 6,520,237; 6,582,518; 6,610,161; 6,613,146, 6,652,693, 6,719,846 and 6,737,102. The adhesive used may be a hot-melt type adhesive having elasticity and flexibility making it suitable for attaching pre-strained elastic materials to substrates, such as OMNIMELT BLOCKS 22 H2401F, or ZEROCREEP brands such as AVANCÉ, available from Bostik, Inc., Wauwatosa, Wisconsin.

In certain embodiments, corners of the front and/or rear belt portion may be trimmed off as suggested in FIG. 18B. The corners may be trimmed off along straight lines, or may be trimmed off along trim paths that are curved and either concave or convex with respect to the remaining area of the belt portion, as may be desired to impart a particular curved leg edge profile. In conjunction with such trimming and the configuration of elastic strands described above, it may be desired to impart bonding between layers along edges of the respective belt portion 322, 323. Such bonding may serve to prevent any separation of the layers along edges that may contribute to creating a ragged appearance, and may also help the rear belt portion more effectively draw inward laterally toward the central chassis 120, under the contractive force of the elastic strands below seams 324. Bonding may be effected by mechanical/compression bonds as described in, for example, U.S. Pat. Nos. 4,854,984 and 4,919,738, by thermal bonds or welds, or by deposits of adhesive between layers. In nonlimiting examples, such bonding may form a pattern along edges. Such bonding may be supplemental to any bonding between layers generally holding the respective belt portion 322, 23 together as a laminate structure.

Side seams 324 may be permanent or refastenable. Permanent seams may be formed between the front belt portion and the rear belt portion by any bonding mechanism wherein the front and rear belt portions may not be forcibly separated without substantial damage to one or both of the front and rear belt portions, or without any included mechanism by which substantial reattachment or refastening may be effected. Bonding forming permanent seams may include compression bonding, thermal bonding/welds, ultrasonic bonding, or adhesive bonding. Refastenable seams may be formed between the front belt portion and the rear belt portion by any mechanism configured to permit substantially non-destructive forcible separation of the front and rear belt portions, and subsequent substantial reattachment or refastening at the same locations. One example of such mechanism is a hook-and-loop fastening system, for example, a VELCRO fastening system. A suitably sized and shaped hooks component may be bonded to one of the front or rear belt portions along the longitudinal edges thereof, and a suitably sized and shaped loops component may be bonded to the other of the front or rear belt portions along the longitudinal edges thereof, in positions in which they may be brought together and engaged to form seams 224. Examples are depicted in U.S. Pat. App. Ser. Nos. 61/787,416; 61/787,332; 61/666,065.

Exemplary belt and absorbent pant constructions are disclosed in U.S. patent application Ser. No. 14/598,783 and Ser. No. 14/032,595.

### Package

The absorbent articles 100 of the present disclosure may be placed into packages. The packages may include polymeric films and/or other materials. Graphics and/or indicia relating to properties of the absorbent articles may be formed on, printed on, positioned on, and/or placed on outer portions of the packages. Each package may include a plurality of absorbent articles. The absorbent articles may be packed under compression so as to reduce the size of the packages, while still providing an adequate amount of absorbent articles per package. By packaging the absorbent articles under compression, caregivers can easily handle and store the packages, while also providing distribution savings to manufacturers owing to the size of the packages.

Accordingly, packages of the absorbent articles of the present disclosure may have an In-Bag Stack Height of less than about 110 mm, less than about 105 mm, less than about 100 mm, less than about 95 mm, less than about 90 mm, less than about 85 mm, less than about 80 mm, less than about 78 mm, less than about 76 mm, less than about 74 mm, less than about 72 mm, or less than about 70 mm, according to the In-Bag Stack Height Test described herein. Alternatively, packages of the absorbent articles of the present disclosure may have an In-Bag Stack Height of from about 70 mm to about 110 mm, from about 70 mm to about 105 mm, from about 70 mm to about 100 mm, from about 70 mm to about 95 mm, from about 70 mm to about 90 mm, from about 70 mm to about 85 mm, from about 72 mm to about 80 mm, or from about 74 mm to about 78 mm, according to the In-Back Stack Height Test described herein.

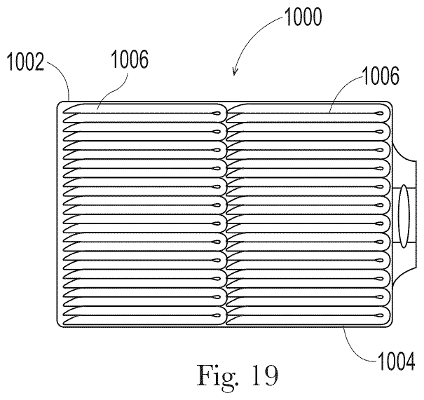

FIG. 19 illustrates an example package 1000 including a plurality of absorbent articles 1004. The package 1000 defines an interior space 1002 in which the plurality of absorbent articles 1004 are situated. The plurality of absorbent articles 1004 are arranged in one or more stacks 1006.

### Test Methods

### Bond Measurement Test Method

The Bond Measurement Test Method is performed on reflected light microscopy images generated using a stereo light microscope (such as Zeiss V20 Stereoscope) and attached camera (such as the Carl Zeiss AxioCam MRc5). The image, containing at least one single repeat unit of a bond impression pattern, is acquired while the sample is fully stretched and backed with a black background. If the area of a single repeat pattern is too large for stereoscope imaging, a DSLR Camera (such as Pentax R20D), or scanner (such as Epson Perfection V750 Pro Flatbed Scanner), capable of at least a 50 micron per pixel resolution may be used to collect the image. Measurements are performed using image analysis software (such as Image Pro Plus software Version 7.0.0.591, Media Cybernetics, USA) calibrated such that distances within the image can be measured precisely to the nearest 50 microns. For purposes of this method, a bond impression is the intentional joining of two or more layers and is the deformed area caused during the bonding process (e.g., the reduced caliper at the site of bonding). Precondition samples at about 23° C.±2 C.° and about 50%±2% relative humidity for 2 hours prior to testing under the same environmental conditions.

Prior to and during image acquisition the sample is fully stretched and secured in a planar extended state. For corrugated laminates, the specimen is fully stretched when the corrugations are substantially flattened by extending the laminate while making sure that the inelastic substrates of the laminate are not plastically deformed. For laminates without corrugations, the specimen is considered fully stretched without such extension.

### Percent Bond Area

Open the image in the image analysis software and identify a single repeat unit within the pattern of bond impressions and unbonded areas. Draw a region of interest (ROI) that encompasses the single repeat unit. Calculate and record the area of the single repeat unit ROI. Next, with the irregular area tool, trace the perimeters of each individual discrete bond impression or portion thereof enclosed within the single repeat unit ROI and record their individual areas. Calculate the sum of all the individual discrete bond impression areas or portions thereof that are within the single repeat unit. Calculate Percent Bond Area as follows:

$$Percent Bond Area ( \% ) = \frac{Sum of Indivdual Discrete Bond Areas}{S ingle Repeat Unit Area} \times 100$$

Repeat this procedure for a total of 3 replicate repeat units. Calculate the arithmetic mean of the 3 values and report as Percent Bond Area to the nearest 0.1%.

Calculate the arithmetic mean of all the recorded whole, exclude any partial, individual discrete bond impression areas and report as the Discrete Bond Area to the nearest 0.01 mm².

### Bond Separation Distance

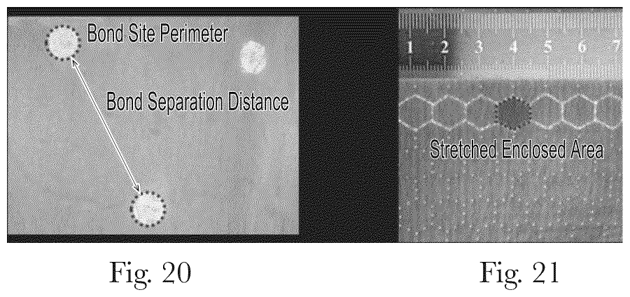

The Bond Separation Distance is defined as the shortest (minimum), straight-line distance between the perimeters of any two individual bond sites within a single closed cell unit (For example, see FIG. 20). Using image analysis software measure and record the Bond Separation Distance for all the discrete bond sites making up the perimeter of an identified single closed cell unit. Repeat this procedure for a total of 3 replicate closed cell units. Calculate and report the arithmetic mean of the recorded values and report as the Bond Separation Distance to the nearest 0.1 mm.

### Stretched Enclosed Area

The Stretched Enclosed Area is defined as the area within a closed cell unit when the sample is in the fully stretched state. In the image identify a closed cell unit and using the image analysis software outline the perimeter of the enclosed cell. The perimeter of the closed unit cell is drawn by connecting the discrete bonds making up the enclosed cell. (For example, see FIG. 21) Measure and record the area of the identified closed cell unit. Repeat this procedure for a total of 3 replicate closed cell units. Calculate and report the arithmetic mean of the 3 values and report as the Stretched Enclosed Area to the nearest whole mm².

### Extensibility Ratio Test Method

While the sample is in its relaxed state use a fine tip pen/marker to draw two parallel lines perpendicular to the primary direction of stretch that indicate the edges of the extensible portion of the substrate. Measure the distance between the drawn parallel lines and record as the relaxed transverse width (W₀) of the extensible portion of the substrate.

Grip the sample outside the extensible region as defined by the drawn lines and stretch the sample in the transverse direction on a flat surface until it is fully stretched out. Once the sample is in the fully stretched state secure it in place.

For corrugated laminates, the sample is fully stretched when the corrugations are substantially flattened by extending the laminate while making sure that the inelastic substrates of the laminate are not plastically deformed.

Measure the distance between the drawn parallel lines and record as the stretched transverse width (W_{S}) of the extensible portion of the substrate.

The Extensibility Ratio is defined to be the ratio W_{S}/W₀.

Repeat this procedure on 3 substantially similar replicate samples. Calculate and report the arithmetic mean of the 3 replicate values and report as the Extensibility Ratio to the nearest tenth.

### Relaxed Enclosed Area

The Relaxed Enclosed Area is calculated by dividing the Stretched Enclosed Area value, obtained from the Bond Measurement Test Method, by the Extensibility Ratio value. The calculated Relaxed Enclosed Area value is reported to the nearest whole mm².

### Hysteresis Test Method

The Hysteresis Test can be used to various specified strain or load values. The Hysteresis Test utilizes a commercial tensile tester (e.g., from Instron Engineering Corp. (Canton, MA), SINTECH-MTS Systems Corporation (Eden Prairie, MN) or equivalent) interfaced with a computer. The computer is used to control the test speed and other test parameters and for collecting, calculating, and reporting the data. The tests are performed under laboratory conditions of 23° C.±2° C. and relative humidity of 50%±2%. The specimens are conditioned for 24 hours prior to testing.

The specimen is cut to dimensions listed in the table below for the test performed. When collecting laminate sample from the web or product, care should be taken to collect specimen with one bond pattern that is being evaluated. If sample can't be cut without having two bond patterns, sample should be tested in a way that only single bond pattern is in the gauge length.

### Test Protocol

1. Select the appropriate grips and load cell. The grips must have one flat surface and must be wide enough to grasp the specimen along its full width. Also, the grips should provide adequate force and suitable surface to ensure that the specimen does not slip during testing. The grips are air actuated grips designed to concentrate the entire gripping force along a single line perpendicular to the direction of testing stress having one flat surface and an opposing face from which protrudes a half round (radius=6 mm, e.g., part number: 56-163-827 from MTS Systems Corp.) or equivalent grips, to minimize slippage of the specimen. The load cell is selected so that the tensile response from the specimen tested is between 25% and 75% of the capacity of the load cell used. Calibrate the tester according to the manufacturer's instructions.

2. Set the distance between the grips (gauge length) as per the test performed (table below).

3. Place the specimen in the flat surfaces of the grips such that the uniform width lies along a direction perpendicular to the gauge length direction. Sample is mounted in a way that sample stretch direction is the test direction. Secure the specimen in the upper grip, let the specimen hang slack, then close the lower grip.

4 Pre-load: Set the slack pre-load at 0.05N, and pre-load crosshead speed of 13 mm/min. This means that the data collection starts when the slack is removed (at a constant crosshead speed of 13 mm/min) with a force of 0.05N. Strain is calculated based on the adjusted gauge length (lᵢₙᵢ), which is the length of the specimen in between the grips of the tensile tester at a force of 0.05N. This adjusted gauge length is taken as the initial specimen length, and it corresponds to a strain of 0%. Percent strain at any point in the test is defined as the change in length relative to the adjusted gauge length, divided by the adjusted gauge length, multiplied by 100.

5(a). First cycle loading: Pull the specimen to the given load or strain at a constant cross head speed as defined in the table below for the test. Report the stretched specimen length between the grips as lmax.

5(b). First cycle unloading: Hold the specimen at the load or strain of step 5(a) for 30 seconds and then return the crosshead to its starting position (0% strain or initial sample length, lᵢₙᵢ) at a constant cross head speed defined in step 5(a) above.

5(c). Hold the specimen in the unstrained state for 1 minute.

5(d). Second cycle: Repeat Step 5(a) and 5(b).:

| | Elastic and | Laminate |
|---|---|---|
| | Extensible Tests | Performance Test |
| Sample Length in the laminate | larger than gage | >26 |
| stretch direction (mm) | length | |
| Sample Width, perpendicular to | 25.4 preferred | 25.4 |
| laminate stretch direction (mm) | (10 mm | |
| | minimum) | |
| Gauge Length (mm) | Minimum 7 | 25.4 |
| | mm, maximum | |
| | 25.4 mm | |
| Test Speed (in/min) | 10 | 10 |
| Load for Step 5(a) | N/A | 4 N |
| Strain % for Step 5(a) | 50% | N/A |

A computer data system records the force exerted on the sample during the test as a function of applied strain. From the resulting data generated, the following quantities are collected and reported.
i. Length of specimen between the grips at a slack preload of 0.05N (lᵢₙᵢ) to the nearest 0.001 mm.
ii. Length of specimen between the grips on first cycle at the at a given strain (lₘₐₓ) to the nearest 0.001 mm.
iii. Length of specimen between the grips at a second cycle load force of 0.07N (lₑₓₜ) to the nearest 0.001 mm.
iv. Force at 50% strain during the first load cycle to the nearest 0.01 N/in (reported as Load Force at 50%) for Laminate Performance Test set-up.
v. Force at 50% strain during the second unload cycle to the nearest 0.01 N/in (reported as Unload Force at 50%) for Laminate Performance Test set-up.

% Set is defined as (lₑₓₜ−lᵢₙᵢ)/(lₘₐₓ−lᵢₙᵢ)*100% to the nearest 0.01%.

The testing is repeated for three separate samples and the average and standard deviation reported.

### Air Permeability Test Method

The air permeability of a laminate or substrate (e.g., film, nonwoven, or article component) is determined by measuring the flow rate of standard conditioned air through a test specimen driven by a specified pressure drop. This test is particularly suited to materials having relatively high permeability to gases, such as nonwovens, apertured ear laminates and the like. ASTM D737 is used, modified as follows.

A TexTest FX 3300 instrument or equivalent is used, available from Textest AG, Switzerland, or from Advanced Testing Instruments ATI in Spartanburg SC, USA. The procedures described in the Operating Instructions for the TEXTEST FX 3300 Air Permeability Tester manual for the Air Tightness Test and the Function and Calibration Check are followed. If a different instrument is used, similar provisions for air tightness and calibration are made according to the manufacturer's instructions.

The specimen is tested while in a relaxed state. Care should be taken to prepare sample with test pattern and eliminate second pattern, if present.

The test pressure drop is set to 500 Pascal and the 1 cm² area test head (model FX 3300-5) or equivalent is used. The result is recorded to three significant digits. The average of 5 specimens is calculated and reported as the Breathability Value (m³/m²/min).

### In-Bag Stack Height Test Method

The in-bag stack height of a package of absorbent articles is determined as follows:

### Equipment

A thickness tester with a flat, rigid horizontal sliding plate is used. The thickness tester is configured so that the horizontal sliding plate moves freely in a vertical direction with the horizontal sliding plate always maintained in a horizontal orientation directly above a flat, rigid horizontal base plate. The thickness tester includes a suitable device for measuring the gap between the horizontal sliding plate and the horizontal base plate to within ±0.5 mm. The horizontal sliding plate and the horizontal base plate are larger than the surface of the absorbent article package that contacts each plate, i.e., each plate extends past the contact surface of the absorbent article package in all directions. The horizontal sliding plate exerts a downward force of 850±1 gram-force (8.34 N) on the absorbent article package, which may be achieved by placing a suitable weight on the center of the non-package-contacting top surface of the horizontal sliding plate so that the total mass of the sliding plate plus added weight is 850±1 grams.

### Test Procedure

Absorbent article packages are equilibrated at 23±2° C. and 50±5% relative humidity prior to measurement.

The horizontal sliding plate is raised and an absorbent article package is placed centrally under the horizontal sliding plate in such a way that the absorbent articles within the package are in a horizontal orientation (see FIG. 19). Any handle or other packaging feature on the surfaces of the package that would contact either of the plates is folded flat against the surface of the package so as to minimize their impact on the measurement. The horizontal sliding plate is lowered slowly until it contacts the top surface of the package and then released. The gap between the horizontal plates is measured to within ±0.5 mm ten seconds after releasing the horizontal sliding plate. Five identical packages (same size packages and same absorbent articles counts) are measured and the arithmetic mean is reported as the package width. The “In-Bag Stack Height”=(package width/absorbent article count per stack)×10 is calculated and reported to within ±0.5 mm.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.” Further, for all numerical ranges specified in the application it is to be appreciated that the range includes all numerical increments within the recited ranges and all ranges formed therein or thereby. For example, a numerical increment may be 0.1 mm, 1 gsm, and/or 0.1 mm².

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising:
a first waist region, a second waist region, and a crotch region disposed between the first and second waist regions;
a chassis comprising a topsheet, a backsheet, and an absorbent core disposed between the topsheet and the backsheet; and
an elastic laminate joined to the chassis in one of the first or second waist regions, wherein the elastic laminate comprises an ultrasonically bonded laminate having:
a first bond pattern comprising a plurality of first repeating units, each first repeating unit comprising a first closed cell unit, wherein bonds in the first bond pattern comprise a Bond Separation Distance of 3.5 mm or less, and
a second bond pattern, wherein the second bond pattern is in non-overlapping relationship with the first bond pattern.

2. The absorbent article of claim 1, wherein the first bond pattern comprises a first Percent Bond Area of at least 3%.

3. The absorbent article of claim 1, wherein the Bond Separation Distance is 3 mm or less.

4. The absorbent article of claim 1, wherein the elastic laminate is elastic in the lateral direction.

5. The absorbent article of claim 1, wherein the first closed cell unit is selected from the group consisting of: polygons, hearts, circles, ellipses, and combinations thereof.

6. The absorbent article of claim 1, wherein the first closed cell comprises a perimeter formed of at least 10 bonds.

7. The absorbent article of claim 1, wherein the first closed cell comprises a Stretched Enclosed Area to Relaxed Enclosed Area ratio of at least about 1.2.

8. The absorbent article of claim 1, wherein the first bond pattern comprises a plurality of second repeating units.

9. The absorbent article of claim 8, wherein the plurality of second repeating units comprise a second closed cell unit.

10. The absorbent article of claim 1, wherein the second bond pattern is void of closed cell units.

11. The absorbent article of claim 1, wherein the second bond pattern comprises a second Percent Bond Area, wherein the second Percent Bond Area is at least 3%.

12. The absorbent article of claim 1, wherein the second bond pattern at least partially overlaps an unstretched region of the elastic laminate, a reinforcement region, a fastening system, or combinations thereof.

13. An absorbent article comprising:
a first waist region, a second waist region, and a crotch region disposed between the first and second waist regions;
a chassis comprising a topsheet, a backsheet, and an absorbent core disposed between the topsheet and the backsheet; and
an elastic laminate joined to the chassis in one of the first or second waist regions,
wherein the elastic laminate comprises an ultrasonically bonded laminate having:
a first coverstock layer, a second coverstock layer and an elastic film disposed between the first and second coverstock layers;
a first bond pattern comprising a plurality of first repeating units, each first repeating unit comprising a first closed cell unit having a first ratio of a Stretched Enclosed Area to a Relaxed Enclosed Area on the first external surface of at least 1.2, and
a second bond pattern, wherein the second bond pattern is in non-overlapping relationship with the first bond pattern.

14. The absorbent article of claim 13, wherein bonds in the first bond pattern comprise a Bond Separation Distance of 3.5 mm or less.

15. The absorbent article of claim 13, comprising a second closed cell unit comprising a second ratio of a Stretched Enclosed Area to a Relaxed Enclosed Area that is less than the first ratio of the Stretched Enclosed Area to the Relaxed Enclosed Area.

16. The absorbent article of claim 13, wherein the second bond pattern comprises a Percent Bond Area that is at least 3%.

17. The absorbent article of claim 16, wherein the second bond pattern at least partially overlaps an unstretched region of the elastic laminate, an inelastic region, a reinforcement region, a fastening system or combinations thereof.

18. An absorbent article comprising:
a first waist region, a second waist region, and a crotch region disposed between the first and second waist regions;
a chassis comprising a topsheet, a backsheet, and an absorbent core disposed between the topsheet and the backsheet; and
an elastic laminate joined to the chassis in one of the first or second waist regions, wherein the elastic laminate comprises an ultrasonically bonded laminate having:
a primary substrate having a first external surface and an opposing first internal surface, a secondary substrate having a second external surface and an opposing second internal surface and an elastomeric film disposed between the primary and secondary substrates;
a first bond pattern comprising a Percent Bond Area of at least 3% disposed in a first region, the first bond pattern comprising a plurality of closed cell units having a Stretched Enclosed Area to Relaxed Enclosed Area ratio of at least 1.2; and
a second bond pattern disposed in a second region, wherein the first and second regions are non-overlapping, and wherein the first bond pattern is non-overlapping relationship to the first bond pattern.

19. The absorbent article of claim 18, wherein the second bond pattern at least partially overlaps an unstretched region of the elastic laminate, an inelastic region, a reinforcement region, a fastening system or combinations thereof.

20. The absorbent article of claim 18, wherein the second bond pattern comprises a Percent Bond Area that is least 3%.
